(19) United States
(12) Patent Application Publication
(10) Pub. No.: US 2025/0201407 A1
(43) Pub. Date: Jun. 19, 2025

(54) PREDICTION OF A REPRESENTATION OF AN EXAMINATION AREA OF AN EXAMINATION OBJECT IN A STATE OF A SEQUENCE OF STATES

(71) Applicant: BAYER AKTIENGESELLSCHAFT, Leverkusen (DE)

(72) Inventors: Matthias LENGA, Leverkusen (DE); Gunnar SCHÜTZ, Leverkusen (DE); Stephan VÖLKENING, Monheim am Rhein (DE)

(21) Appl. No.: 18/840,591

(22) PCT Filed: Feb. 10, 2023
(86) PCT No.: PCT/EP2023/053323

§ 371 Date: Aug. 22, 2024

(30) Foreign Application Priority Data
Feb. 24, 2022 (EP) 22158527.6

(51) Int. Cl. G16H 50/20; G06V 10/25; G06V 10/774; G06V 10/776; G06V 20/50; G16H 30/40

(52) U.S. Cl. CPC G16H 50/20; G06V 10/25; G06V 10/774; G06V 10/776; G06V 20/50; G16H 30/40; G06V 2201/031

(57) ABSTRACT

The present invention relates to the technical field of radiology, and in particular to assisting radiologists in radiological examinations using artificial intelligence methods. The present invention also relates to training a machine learning model and using the trained model to predict representations of an examination area in one or more states of a sequence of states in a radiological examination.

The present invention relates to the technical field of radiology, in particular that of assisting radiologists in radiological examinations using artificial intelligence methods. The present invention is concerned with the training of a machine-learning model and the utilization of the trained model for predicting representations of an examination region in one or more states of a sequence of states in a radiological examination.

Tracking processes over time within the body of a human or animal using imaging methods plays an important role, inter alia, in the diagnosis and/or therapy of diseases.

An example that may be mentioned is the detection and differential diagnosis of focal liver lesions by means of dynamic contrast-enhanced magnetic resonance imaging (MRI) using a hepatobiliary contrast agent.

A hepatobiliary contrast agent such as Primovist® can be used for the detection of tumors in the liver. Blood is supplied to healthy liver tissue primarily via the portal vein (vena portae), whereas most primary tumours are supplied by the liver artery (arteria hepatica). After intravenous injection of a bolus of contrast agent, it is accordingly possible to observe a time delay between the signal increase in the healthy liver parenchyma and in the tumor.

Besides malignant tumors, what are commonly found in the liver are benign lesions such as cysts, hemangiomas and focal nodular hyperplasias (FNH). A proper planning of therapy requires that these be differentiated from the malignant tumours. Primovist® can be used for the identification of benign and malignant focal liver lesions. By means of T1-weighted MRI, it provides information about the character of said lesions. Differentiation is achieved by utilizing the difference in blood supply to liver and tumor and the time profile of contrast enhancement.

In the case of the contrast enhancement achieved by Primovist® during the wash-in phase, what are observed are typical perfusion patterns which provide information for the characterization of the lesions. Depicting the vascularization helps to characterize the lesion types and to determine the spatial relationship between tumor and blood vessels.

In the case of T1-weighted MRI images, Primovist® leads, 10-20 minutes after the injection (in the hepatobiliary phase), to a distinct signal enhancement in the healthy liver parenchyma, whereas lesions containing no hepatocytes or only a few hepatocytes, for example metastases or moderately to poorly differentiated hepatocellular carcinomas (HCCs), appear as darker regions.

Tracking the spread of the contrast agent over time thus provides a good way of detecting and differentially diagnosing focal liver lesions; however, the examination extends over a comparatively long period of time. Over this period of time, movements by the patient should be largely avoided in order to minimize movement artifacts in the MRI images. The lengthy restriction of movement can be unpleasant for a patient.

In the laid-open application WO2021/052896A1, it is proposed that one or more MRI images during the hepatobiliary phase not be generated by measurement, but that they be calculated (predicted) on the basis of MRI images from one or more preceding phases in order to shorten the time spent by the patient in the MRI scanner.

The approach described in the laid-open application WO2021/052896A1 involves training a machine-learning model to use MRI images of an examination region before and/or immediately after the administration of a contrast agent as a basis to predict an MRI image of the examination region at a later time. The model is thus trained to map multiple MRI images as input data onto an MRI image as output data.

Proceeding from the described prior art, it is an object of the invention to improve the quality of prediction of the machine-learning model and/or to create a model that learns the dynamics of the spread of contrast agent in the examination region in order to be able to use it in a variety of ways.

This object is achieved by the subjects of the present independent claims. Preferred embodiments can be found in the dependent claims and also in the present description and the drawings.

The invention provides in a first aspect a computer-implemented method for training a machine-learning model. The training method comprises:
receiving training data,
   where the training data comprise representations ^{T}R₁ to ^{T}Rₙ of an examination region for a multiplicity of examination objects, where n is an integer greater than two,
   where each representation ^{T}Rᵢ represents the examination region in one state Zᵢ of a sequence of states Z₁ to Zₙ, where i is an index passing through the integers from 1 to n,
training the machine-learning model,
   where the model is trained to generate, starting from the representation ^{T}R₁, representations ^{T}R₂* to ^{T}Rₙ* one after the other for each examination object of the multiplicity of examination objects, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n, where the representation ^{T}Rₖ* represents the examination region in the state Zₖ and the representation ^{T}Rₖ₋₁* represents the examination region in the state Zₖ₋₁, and the state Zₖ₋₁ directly precedes the state Zₖ,
   where differences between the generated representations ^{T}Rⱼ* and the representations ^{T}Rⱼ are quantified by means of a loss function, where j is an index passing through the numbers from 2 to n,
   where the differences are minimized in an optimization method by modifying model parameters of the machine-learning model,
storing the trained machine-learning model and/or utilizing the machine-learning model for prediction.

The invention further provides a computer-implemented method for predicting one or more representations of an examination region of an examination object. The prediction method comprises:
receiving a representation Rₚ of the examination region of the examination object,
   where the representation Rₚ represents the examination region in one state Zₚ of a sequence of states Z₁ to Zₙ,
   where p is an integer less than n,
   where n is an integer greater than two,
feeding the representation Rₚ to a trained machine-learning model,
   where the trained machine-learning model has been trained on the basis of training data to generate, starting from a first representation ^{T}R₁, a number n−1 of representations ^{T}R₂* to ^{T}Rₙ* one after the other,
   where the first representation ^{T}R₁ represents the examination region in the first state Z₁ and each generated representation ^{T}Rⱼ* represents the examination region in the state Zⱼ, where j is an index passing through the numbers from 2 to n,
   where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n,
receiving one or more representations R_{p+q}* of the examination region from the machine-learning model,
   where each of the one or more representations R_{p+q}* represents the examination region in the state Z_{p+q}, where q is an index passing through the numbers from 1 to m, where m is an integer less than n−1 or equal to n−1 or greater than n−1,
outputting and/or storing and/or transmitting the one or more representations R_{p+q}*.

The present invention further provides a computer system comprising
an input unit,
a control and calculation unit and
an output unit,
   wherein the control and calculation unit is configured
to cause the input unit to receive a representation Rₚ of an examination region of an examination object, where the representation Rₚ represents the examination region in one state Zₚ of a sequence of states Z₁ to Zₙ, where p is an integer less than n, where n is an integer greater than two,
to feed the representation Rₚ to a trained machine-learning model, where the trained machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region in the first state Z₁ and each further generated representation ^{T}Rⱼ* represents the examination region in the state Zⱼ, where j is an index passing through the numbers from 2 to n, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n,
to receive from the machine-learning model one or more representations R_{p+q}* of the examination region, where each of the one or more representations R_{p+q}* represents the examination region in the state Z_{p+q}, where q is an index passing through the numbers from 1 to m, where m is an integer less than n−1 or equal to n−1 or greater than n−1,
to cause the output unit to output the one or more representations R_{p+q}* and/or to store them and/or to transmit them to a separate computer system.

The present invention further provides a computer program product comprising a data memory in which there is stored a computer program that can be loaded into a working memory of a computer system, where it causes the computer system to execute the following steps:
receiving a representation Rₚ of an examination region of an examination object, where the representation Rₚ represents the examination region in one state Zₚ of a sequence of states Z₁ to Zₙ, where p is an integer less than n, where n is an integer greater than two,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region in the first state Z₁ and each generated representation ^{T}Rⱼ* represents the examination region in the state Zⱼ, where j is an index passing through the numbers from 2 to n, where the representation ^{T}R₂* is generated at least partly on the basis of the representation ^{T}R₁ and each further representation ^{T}Rₖ* is generated at least partly on the basis of a respective previously generated representation ^{T}Rₖ₋₁*, where k is an index passing through the numbers from 3 to n,
receiving one or more representations R_{p+q}* of the examination region from the machine-learning model, where each of the one or more representations R_{p+q}* represents the examination region in the state Z_{p+q}, where q is an index passing through the numbers from 1 to m, where m is an integer less than n−1 or equal to n−1 or greater than n−1,
outputting and/or storing and/or transmitting the one or more representations R_{p+q}*.

The present invention further provides for a use of a contrast agent in a radiological examination method, wherein the radiological examination method comprises the following steps:
receiving a representation Rₚ of an examination region of an examination object, where the representation Rₚ represents the examination region before or after the administration of the contrast agent in one state Zₚ of a sequence of states Z₁ to Zₙ, where p is an integer less than n, where n is an integer greater than two,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region in the first state Z₁ and each generated representation ^{T}Rⱼ* represents the examination region in the state Zⱼ, where j is an index passing through the numbers from 2 to n, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n,
receiving one or more representations R_{p+q}* of the examination region from the machine-learning model, where each of the one or more representations R_{p+q}* represents the examination region in one state Z_{p+q}, where q is an index passing through the numbers from 1 to m, where m is an integer less than n−1 or equal to n−1 or greater than n−1,
outputting and/or storing and/or transmitting the one or more representations R_{p+q}*.

The present invention further provides a contrast agent for use in a radiological examination method, wherein the radiological examination method comprises the following steps:
generating a representation Rₚ of an examination region of an examination object, where the representation Rₚ represents the examination region before or after the administration of the contrast agent in one state Zₚ of a sequence of states Z₁ to Zₙ, where p is an integer less than n, where n is an integer greater than two,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region in the first state Z₁ and each generated representation ^{T}Rⱼ* represents the examination region in the state Zⱼ, where j is an index passing through the numbers from 2 to n, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n,
receiving one or more representations R_{p+q}* of the examination region from the machine-learning model, where each of the one or more representations R_{p+q}* represents the examination region in the state Z_{p+q}, where q is an index passing through the numbers from 1 to m, where m is an integer less than n−1 or equal to n−1 or greater than n−1,
outputting and/or storing and/or transmitting the one or more representations R_{p+q}*.

The present invention further provides a kit comprising a contrast agent and a computer program product, wherein the computer program product comprises a computer program that can be loaded into a working memory of a computer system, where it causes the computer system to execute the following steps:
1 receiving a representation Rₚ of an examination region of an examination object, where the representation Rₚ represents the examination region before or after the administration of the contrast agent in one state Zₚ of a sequence of states Z₁ to Zₙ, where p is an integer less than n, where n is an integer greater than two,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region in the first state Z₁ and each generated representation ^{T}Rⱼ* represents the examination region in the state Zⱼ, where j is an index passing through the numbers from 2 to n, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n,
receiving one or more representations R_{p+q}* of the examination region from the machine-learning model, where each of the one or more representations R_{p+q}* represents the examination region in the state Z_{p+q}, where q is an index passing through the numbers from 1 to m, where m is an integer less than n−1 or equal to n−1 or greater than n−1,
outputting and/or storing and/or transmitting the one or more representations R_{p+q}*.

The invention will be more particularly elucidated below without distinguishing between the subjects of the invention (training method, prediction method, computer system, computer program product, use, contrast agent for use, kit). Rather, the elucidations that follow are intended to apply analogously to all subjects of the invention, irrespective of the context (training method, prediction method, computer system, computer program product, use, contrast agent for use, kit) in which they occur.

Where steps are stated in an order in the present description or in the claims, this does not necessarily mean that the invention is limited to the order stated. Instead, it is conceivable that the steps are also executed in a different order or else in parallel with one another, the exception being when one step builds on another step, thereby making it imperative that the step building on the previous step be executed next (which will however become clear in the individual case). The orders stated are thus preferred embodiments of the invention.

With the aid of the present invention, representations of an examination region of an examination object can be predicted.

The “examination object” is normally a living being, preferably a mammal, most preferably a human.

The “examination region” is part of the examination object, for example an organ or part of an organ, such as the liver, brain, heart, kidney, lung, stomach, intestines or part of the aforementioned organs, or multiple organs or another part of the body.

In a preferred embodiment of the present invention, the examination region is the liver or part of the liver of a human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

A “representation of the examination region” is preferably a medical image. A representation of the examination region is preferably the result of a radiological examination.

“Radiology” is the branch of medicine that is concerned with the use of predominantly electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present invention thus encompasses in particular the following examination methods: computed tomography, magnetic resonance imaging, sonography.

In a preferred embodiment of the present invention, the radiological examination is a computed tomography examination or a magnetic resonance imaging examination.

Computed tomography (CT) is an X-ray method which depicts the human body in cross-sectional images (sectional imaging method). Compared to a conventional X-ray image, on which usually only coarse structures and bones are identifiable, CT images also capture in detail soft tissues with small differences in contrast. An X-ray tube generates a so-called X-ray fan beam, which penetrates the body and is attenuated to varying degrees within the body owing to the various structures, such as organs and bones. The receiving detectors opposite the X-ray emitter receive the signals of varying strength and forward them to a computer, which puts together cross-sectional images of the body from the received data. Computed tomography images (CT images) can be observed in 2D or else in 3D. For better differentiability of structures within the body of the human (e.g., vessels), a contrast agent can be injected into, for example, a vein before CT images are generated.

Magnetic resonance imaging, MRI for short, is an imaging method that is used especially in medical diagnostics for depicting structure and function of tissues and organs in the human or animal body.

In MRI, the magnetic moments of protons in an examination region are aligned in a basic magnetic field, with the result that there is a macroscopic magnetization along a longitudinal direction. This is then deflected from the resting position by irradiation with high-frequency (HF) pulses (excitation). The return of the excited states to the resting position (relaxation), or magnetization dynamics, is then detected as relaxation signals by means of one or more HF receiver coils.

For spatial encoding, rapidly switched magnetic gradient fields are superimposed on the basic magnetic field. The captured relaxation signals, or detected MRI data, are initially present as raw data in frequency space, and can be transformed by subsequent inverse Fourier transform into real space (image space).

A representation of the examination region in the context of the present invention can be an MRI image, a computed tomogram, an ultrasound image or the like.

A representation of the examination region in the context of the present invention can be a representation in real space (image space), a representation in frequency space or some other representation. Preferably, representations of the examination region are present in a real-space depiction or in a form that can be converted (transformed) into a real-space depiction. A representation in real space is often also referred to as a pictorial depiction or as an image.

In a representation in real space, also referred to in this description as real-space depiction or real-space representation, the examination region is normally represented by a large number of image elements (pixels or voxels) that may for example be in a raster arrangement, in which case each image element represents a part of the examination region and each image element may be assigned a color value or gray value. A format widely used in radiology for storing and processing representations in real space is the DICOM format. DICOM (Digital Imaging and Communications in Medicine) is an open standard for storing and exchanging information in medical image data management.

A representation in real space can for example be converted (transformed) by a Fourier transform into a representation in frequency space. Conversely, a representation in frequency space can for example be converted (transformed) by an inverse Fourier transform into a representation in real space.

In a representation in frequency space, also referred to in this description as frequency-space depiction or frequency-space representation, the examination region is represented by a superposition of fundamental frequencies. For example, the examination region may be represented by a sum of sine and/or cosine functions having different amplitudes, frequencies and phases. The amplitudes and phases may be plotted as a function of the frequencies, for example, in a two- or three-dimensional representation. Normally, the lowest frequency (origin) is placed in the center. The further away from this center, the higher the frequencies. Each frequency can be assigned an amplitude representing the frequency in the frequency-space depiction and a phase indicating the extent of the shift of the respective wave with respect to a sine or cosine wave.

Details about real-space depictions and frequency-space depictions and their respective interconversion are described in numerous publications, see for example https://see.stanford.edu/materials/lsoftaee261/book-fall-07.pdf.

A representation in the context of the present invention represents the examination region in one state of a sequence of states.

The sequence of states is preferably a chronological sequence. States directly following one another in a chronological sequence may always have the same time interval between one another or they may have varying time intervals. Mixed forms are also conceivable.

This shall be explained with reference to FIG. 1.

FIG. 1 shows two timelines (t=time): a first timeline (a) and a second timeline (b). The timelines are each labeled with defined time points t₁, t₂, t₃ and t₄. At each time point, the examination region can have a different state, i.e., each time point can represent one state of the examination region.

Time points t₁, t₂, t₃ and to form for each timeline a chronological sequence: t₁→t₂→t₃→t₄. Time point t₂ directly follows time point t₁, and the time interval between t₁ and t₂ is t₂−t₁; time point t₃ directly follows time point t₂, and the time interval between t₂ and t₃ is t₃−t₂; time point t₄ directly follows time point t₃, and the time interval between time points t₃ and t₄ is t₄−t₃.

In the case of the first timeline (a), the time intervals of time points directly following one another are identical for all time points directly following one another: t₂−t₁=t₃−t₂=t₄−t₃.

In the case of the second timeline (b), the time intervals of time points directly following one another vary for all time points directly following one another: t₂−t₁ @t₃−t₂≠t₄−t₃. In the present example, the time intervals increase in the case of the time points directly following one another second time axis (b): t₂−t₁<t₃−t₂<t₄−t₃. However, it is also conceivable that they fall or initially rise and then fall or initially fall and then rise or have some other distribution along the time axis.

It should be noted that the time axis does not necessarily have to run in the direction of increasing time. This means that time point t₁ as seen from time point t₂ does not necessarily have to lie in the past; it is also conceivable that the time axis shows decreasing time and time point t₁ as seen from time point t₂ lies in the future. In other words, if there is a chronological sequence of states, a second state directly following a first state may, as seen from the time point of the first state, lie in the future or lie in the past.

The (preferably chronological) sequence of states may be defined in a first step. The sequence of states defines what training data are used to train the machine-learning model and what representations can be generated (predicted) by the machine-learning model. In other words, what are required for training of the machine-learning model are representations of the examination region of a multiplicity of examination objects in states that are defined by the sequence of states, and the trained machine-learning model can normally only generate representations of states that were part of the training (exceptions to this rule are listed later in the description).

Preferably, the sequence of states defines different states of the examination region before and/or during and/or after single or multiple administration of a contrast agent.

“Contrast agents” are substances or mixtures of substances that improve the depiction of structures and functions of the body in radiological imaging methods.

Examples of contrast agents can be found in the literature (see for example A. S. L. Jascinth et al.: Contrast Agents in computed tomography: A Review, Journal of Applied Dental and Medical Sciences, 2016, vol. 2, issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M. R. Nough et al.: Radiographic and magnetic resonances contrast agents: Essentials and tips for safe practices, World J Radiol. 2017 Sep. 28; 9 (9): 339-349; L. C. Abonyi et al.: Intravascular Contrast Media in Radiography: Historical Development & Review of Risk Factors for Adverse Reactions, South American Journal of Clinical Research, 2016, vol. 3, issue 1, 1-10; ACR Manual on Contrast Media, 2020, ISBN: 978-1-55903-012-0; A. Ignee et al.: Ultrasound contrast agents, Endosc Ultrasound. 2016 November-December; 5 (6): 355-362).

Preferably, the contrast agent is an MRI contrast agent. MRI contrast agents exert their effect by altering the relaxation times of structures that take up contrast agents. A distinction can be made between two groups of substances: paramagnetic and superparamagnetic substances. Both groups of substances have unpaired electrons that induce a magnetic field around the individual atoms or molecules. Superparamagnetic contrast agents result in a predominant shortening of T2, whereas paramagnetic contrast agents mainly result in a shortening of T1. The effect of said contrast agents is indirect, since the contrast agent does not itself emit a signal, but instead merely influences the intensity of signals in its vicinity. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), and gadobutrol (Gadovist®).

Preferably, the MRI contrast agent is a hepatobiliary contrast agent. A hepatobiliary contrast agent has the characteristic features of being specifically taken up by liver cells (hepatocytes), accumulating in the functional tissue (parenchyma) and enhancing contrast in healthy liver tissue. An example of a hepatobiliary contrast agent is the disodium salt of gadoxetic acid (Gd-EOB-DTPA disodium), which is described in U.S. Pat. No. 6,039,931A and is commercially available under the trade names Primovist® and Eovist®.

After the intravenous administration of a hepatobiliary contrast agent in the form of a bolus into an arm vein of a human, the contrast agent reaches the liver first via the arteries. These are depicted with contrast enhancement in the corresponding MRI images. The phase in which the liver arteries are depicted with contrast enhancement in MRI images is referred to as “arterial phase”.

Subsequently, the contrast agent reaches the liver via the liver veins. Whereas the contrast in the liver arteries is already decreasing, the contrast in the liver veins is reaching a maximum. The phase in which the liver veins are depicted with contrast enhancement in MRI images is referred to as “portal venous phase”.

The portal venous phase is followed by the “transitional phase”, in which the contrast in the liver arteries drops further and the contrast in the liver veins likewise drops. When using a hepatobiliary contrast agent, the contrast in the healthy liver cells gradually rises in the transitional phase.

The arterial phase, the portal venous phase and the transitional phase are also referred to collectively as “dynamic phase”.

10-20 minutes after its injection, a hepatobiliary contrast agent leads to a distinct signal enhancement in the healthy liver parenchyma. This phase is referred to as “hepatobiliary phase”. The contrast agent is eliminated only slowly from the liver cells; accordingly, the hepatobiliary phase can last for two hours and longer.

The stated phases are described in greater detail, for example, in the following publications: J. Magn. Reson. Imaging, 2012, 35 (3): 492-511, doi: 10.1002/jmri.22833; Clujul Medical, 2015, Vol. 88 No. 4:438-448, DOI: 10.15386/cjmed-414; Journal of Hepatology, 2019, Vol. 71:534-542, http://dx.doi.org/10.1016/j.jhep.2019.05.005).

The states from the sequence of states may for example be the state of the liver as examination region before administration of a hepatobiliary contrast agent,
in the arterial phase,
in the portal venous phase,
in the transitional phase and/or
in the hepatobiliary phase.

It is conceivable that there are more states or fewer states.

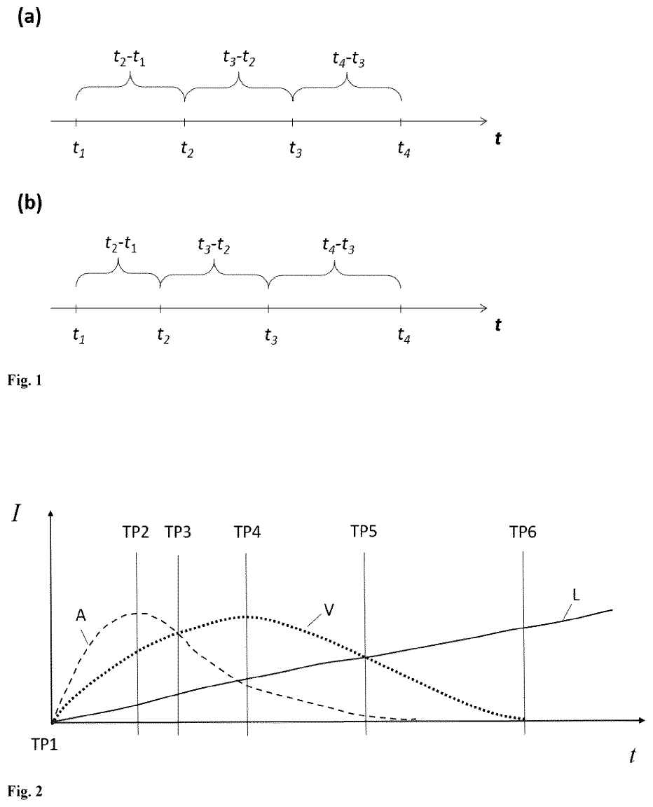

The stated phases are explained in greater detail below with reference to FIG. 2. FIG. 2 shows schematically the temporal profile (t=time) of the signal intensities I that are caused by a hepatobiliary contrast agent in liver arteries A, liver veins V and healthy liver cells L in a dynamic contrast-enhanced MRI examination. The signal intensity I has a positive correlation with the concentration of the contrast agent in the stated regions. Upon an intravenous bolus injection into the arm of a human, the concentration of the contrast agent rises in the liver arteries A first of all (dashed curve). The concentration passes through a maximum and then drops. The concentration in the liver veins V rises more slowly than in the liver arteries and reaches its maximum later (dotted curve). The concentration of the contrast agent in the liver cells L rises slowly (solid curve) and reaches its maximum only at a very much later time point (not depicted in FIG. 2). A few characteristic time points can be defined: At time point TP1, contrast agent is administered intravenously as a bolus. Since the administration of a contrast agent itself requires a certain time span, time point TP1 preferably defines the time point at which the administration is completed, i.e. at which contrast agent is completely introduced into the examination object. At time point TP2, the signal intensity of the contrast agent in the liver arteries A reaches its maximum. At time point TP3, the curves of the signal intensities for the liver arteries A and the liver veins V intersect. At time point TP4, the signal intensity of the contrast agent in the liver veins V passes through its maximum. At time point TP5, the curves of the signal intensities for the liver arteries A and the healthy liver cells L intersect. At time point TP6, the concentrations in the liver arteries A and the liver veins V have dropped to a level at which they no longer cause a measurable contrast enhancement.

The states of the sequence of states may comprise a first state present before time point TP1, a second state present at time point TP2, a third state present at time point TP3, a fourth state present at time point TP4, a fifth state present at time point TP5 and/or a sixth state present at time point TP6 and/or afterwards.

In general,
a first state may be the state of an examination region at a first time point before the administration of a contrast agent,
a second state may be the state of the examination region at a second time point, after the administration of the contrast agent,
a third state may be the state of the examination region at a third time point, after the administration of the contrast agent,
a fourth state may be one state of the examination region at a fourth time point, after the administration of the contrast agent, and so on.

As described above, all time points directly following one another, or a portion thereof, may have a constant time interval between one another and/or they may have a variable time distance between one another.

According to the invention, there are at least three states; preferably, the number of states is between 3 and 100. However, the number of states is not limited to 100.

At each state of the sequence of states, there may be one or more representations representing the examination region in the respective state.

Normally, at a first state there is at least one first representation representing the examination region in the first state, at a second state there is at least one second representation representing the examination region in the second state, at a third state there is at least one third representation representing the examination region in the third state, and so on.

To facilitate understanding of the present description, the present invention is mainly described on the basis of one representation per state; however, this is not to be understood as restricting the invention.

Returning to the example described above, there may be for example
one or more first representations representing a liver before the administration of a hepatobiliary contrast agent,
one or more second representations representing the liver during the arterial phase, one or more third representations representing the liver during the portal venous phase,
one or more fourth representations representing the liver during the transitional phase, and/or
one or more fifth representations representing the liver during the hepatobiliary phase.

With the aid of the present invention, one or more representations of an examination region of an examination object that represent the examination region in one state of a sequence of states can be predicted.

The prediction is done with the aid of a machine-learning model.

A “machine learning model” can be understood as meaning a computer-implemented data processing architecture. The model can receive input data and supply output data on the basis of said input data and model parameters. The model can learn a relationship between the input data and the output data by means of training. During training, model parameters can be adjusted so as to supply a desired output for a particular input.

During the training of such a model, the model is presented with training data from which it can learn. The trained machine learning model is the result of the training process. Besides input data, the training data include the correct output data (target data) that are to be generated by the model on the basis of the input data. During training, patterns that map the input data onto the target data are recognized.

In the training process, the input data of the training data are input into the model, and the model generates output data. The output data are compared with the target data. Model parameters are altered so as to reduce the differences between the output data and the target data to a (defined) minimum.

The differences can be quantified with the aid of a loss function. A loss function of this kind can be used to calculate a loss for a given set of output data and target data. The aim of the training process can consist in altering (adapting) the parameters of the machine learning model so that the loss for all pairs of the training data set is reduced to a (defined) minimum. Adjusting model parameters to reduce loss can be done in an optimization method, such as a gradient method.

For example, if the output data and the target data are numbers, the loss function can be the absolute difference between these numbers. In this case, a high absolute loss value can mean that one or more model parameters need to be altered to a substantial degree.

In the case of output data in the form of vectors, for example, difference metrics between vectors such as the mean squared error, a cosine distance, a norm of the difference vector such as a Euclidean distance, a Chebyshev distance, an Lp norm of a difference vector, a weighted norm or any other type of difference metric of two vectors can be chosen as the loss function.

In the case of higher-dimensional outputs, such as two-dimensional, three-dimensional or higher-dimensional outputs, an element-by-element difference metric can for example be used. Alternatively or in addition, the output data can be transformed, for example into a one-dimensional vector, before a loss is calculated.

In the present case, the machine-learning model is trained on the basis of training data to generate a representation of an examination region in one state of a sequence of states on the basis of representations of the examination region in preceding states of the sequence of states, said model starting from a first state and generating representations of the examination region in subsequent states one after the other (iteratively), each representation of the examination region in each state being generated at least partly on the basis of a (predicted) representation of the preceding state.

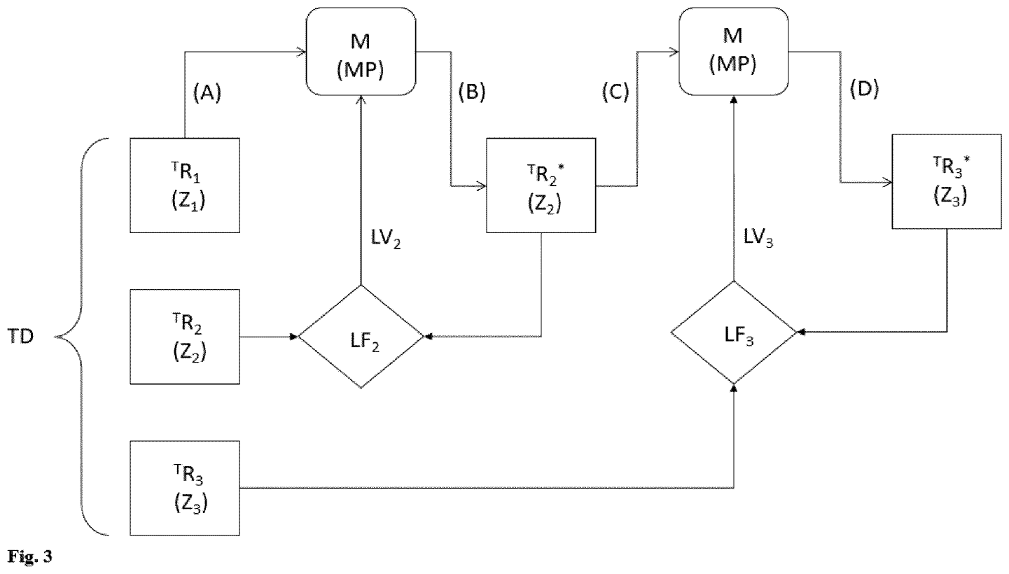

This shall be explained on the basis of an example of three states without any intention of restricting the invention to this embodiment. In this explanation, reference is made to FIG. 3.

It should be noted that representations of the examination region are identified by the letter R in this description. The letter R can be followed by an index, for example R₁, R₂, R₃ or Rᵢ. The index added as a suffix indicates what is represented by the respective representation. The representation R₁ represents the examination region of an examination object in a first state Z₁ of a sequence of states; the representation R₂ represents the examination region of an examination object in a second state Z₂ of the sequence of states; the representation R₃ represents the examination region in a third state Z₃ of the sequence of states, and so on. In general, the representation Rᵢ represents the examination region of an examination object in one state Zᵢ of a sequence of states, where i is an index passing through the integers from i to n, where n is an integer greater than two. The expression “an index i passes through the integers from a to b” means that i assumes the values from a to b one after the other, i.e., it first assumes the value a (i=a), then the value a+1 (i=a+1), and so on until i reaches the value b (i=b). The expression “each representation Rᵢ* represents the examination region in one state Zᵢ of a sequence of states Z₁ to Zₙ” means that the representation R₁* represents the examination region in the state Z₁, the representation R₂* represents the examination region in the state Z₂, and so on, where the states Z₁ to Zₙ form a sequence (in the mathematical sense). Especially in the claims and the drawings, representations which are used for training and representations which are generated in training are identified by a T added as a prefix, for example in the case of ^{T}R₁, ^{T}R₂ and ^{T}Rₖ₋₁*. Especially in the claims and the drawings, representations generated by means of the machine-learning model are identified by a superscript asterisk *, as in the case of R₁*, R₂* and ^{T}Rₖ₋₁*. The labels described herein serve merely for clarification and serve in particular to avoid any objections relating to clarity in the patent grant procedure.

FIG. 3 shows three representations: a first representation ^{T}R₁, a second representation ^{T}R₂ and a third representation ^{T}R₃. The first representation ^{T}R₁ represents an examination region of an examination object in a first state Z₁, the second representation ^{T}R₂ represents the examination region in a second state Z₂ and the third representation ^{T}R₃ represents the examination region in a third state Z₃. The three states form a sequence of states: first state (Z₁)→second state (Z₂)→third state (Z₃).

The three representations ^{T}R₁, ^{T}R₂ and ^{T}R₃ form a data set within training data TD. The training data TD comprise a multiplicity of such data sets. The term “multiplicity” preferably means more than 100. Each data set normally (but not necessarily) comprises three representations of the examination region for the three states, one representation for each state. The examination region is normally always the same and each data set comprises representations of the examination region in the three states, which are likewise normally identical for all data sets: first state, second state, third state. Only the examination object may vary. Each data set normally comes from a different examination object. The statements made in this paragraph are generally applicable, not just in relation to the example shown in FIG. 3.

In a first step (A), the first representation ^{T}R₁ is fed to a machine-learning model M. The machine-learning model M is configured to generate on the basis of the first representation ^{T}R₁ and on the basis of model parameters MP an output ^{T}R₂* (step (B)). The output ^{T}R₂* is intended to approximate the second representation ^{T}R₂ as closely as possible; ideally, the output ^{T}R₂* cannot be distinguished from the second representation ^{T}R₂. In other words, the output ^{T}R₂* is a predicted second representation. The output ^{T}R₂* is compared with the (actual) second representation ^{T}R₂ and the differences are quantified with the aid of a loss function LF₂. In the case of multiple examination objects, a loss value LV₂ can be calculated for each pair of output ^{T}R₂* and second representation ^{T}R₂ by means of the loss function LF₂.

Examples of loss functions that can be used in general (not limited to the example in FIG. 3) to carry out the present invention are L1 loss function, L2 loss function, Lp loss function, structural similarity index measure (SSIM), VGG loss function, perceptual loss function or a combination of the aforementioned functions or other loss functions. Further details on loss functions can be found, for example, in the scientific literature (see for example: R. Mechrez et al.: The Contextual Loss for Image Transformation with Non-Aligned Data, 2018, arXiv: 1803.02077v4; H. Zhao et al.: Loss Functions for Image Restoration with Neural Networks, 2018, arXiv: 1511.08861v3).

The output ^{T}R₂* is refed to the machine-learning model M in step (C). Even though FIG. 3 may suggest otherwise, the machine-learning model M to which the first representation ^{T}R₁ is fed in step (A) and that to which the generated representation ^{T}R₂* is fed in step (C) are the same model. This means that the machine-learning model M is not only configured to generate on the basis of the first representation a predicted second representation, but is also configured to generate from a (predicted and/or actual) second representation an output ^{T}R₃* approximating the third representation ^{T}R₃ as closely as possible. The output ^{T}R₃* is a predicted third representation. The output ^{T}R₃* is compared with the third representation ^{T}R₃. With the aid of a loss function LF₃, the difference between the output ^{T}R₃* and the third representation ^{T}R₃ can be quantified. In the case of multiple examination objects, a loss value LV₃ can be determined for each pair of a third representation ^{T}R₃ and a generated representation ^{T}R₃*.

Preferably, the machine-learning model undergoes end-to-end training. This means that the machine-learning model is simultaneously trained to generate a predicted second representation on the basis of the first representation and a predicted third representation on the basis of the predicted second representation. Preferably, this is achieved by using a loss function which takes into account both the differences between the predicted second representation and the second representation and the differences between the predicted third representation and the third representation.

It is possible to quantify the differences between the second representation and the predicted second representation with the aid of the loss function LF₂ and to quantify the differences between the third representation and the predicted third representation with the aid of the loss function LF₃. A loss function LF taking into account both differences may for example be the sum of the individual loss functions: LF=LF₂+LF₃. It is also possible for the components formed by the individual loss functions LF₂ and LF₃ in the total loss function LF to be weighted differently: LF=w₂·LF₂+w₃·LF₃, where w₂ and w₃ are weight factors that may assume, for example, values between 0 and 1. The value zero may be used for a weight factor, for example if a representation is missing in a data set (further details can be found later in the description).

The training method shown in FIG. 3 is a preferred embodiment of the present disclosure and comprises the steps of:
receiving training data,
   where the training data comprise representations ^{T}R₁ to ^{T}R₃ of an examination region for a multiplicity of examination objects,
   where the representation ^{T}R₁ represents the examination region in a first state Z₁, the representation ^{T}R₂ represents the examination region in a second state Z₂ and the representation ^{T}R₃ represents the examination region in a third state Z₃, where the states Z₁, Z₂ and Z₃ form a sequence of states,
training a machine-learning model, where the training for each examination object of the multiplicity of examination objects comprises:
   feeding the representation ^{T}R₁ to the machine-learning model, where the machine-learning model is configured to generate at least partly on the basis of the representation ^{T}R₁ and on the basis of model parameters a representation ^{T}R_{2*},
   feeding the representation ^{T}R₂* to the machine-learning model, where the machine-learning model is further configured to generate at least partly on the basis of the representation ^{T}R₂* and on the basis of model parameters a representation ^{T}R_{3*},
   quantifying differences between the representation ^{T}R₂ and the generated representation ^{T}R₂* and between the representation ^{T}R₃ and the generated representation ^{T}R_{3*},
   minimizing the differences by modifying the model parameters,
storing the trained machine-learning model and/or using the machine-learning model for prediction.

When the trained machine-learning model is (later) used for prediction of new representations, this may be to predict a third representation on the basis of a first representation. For this purpose, it would be possible in principle to train a model to generate the third representation directly on the basis of the first representation. However, according to the invention, the machine-learning model is trained to generate the third representation on the basis of the first representation not in one step, but in two steps, with a second representation being predicted in a first step, followed by prediction of the third representation on the basis of the second representation. The advantage of the approach according to the invention over the aforementioned “direct training” is, inter alia, that additional training data (second representations representing the examination region in the second state) can be used, thus making it possible to achieve a higher accuracy of prediction. Furthermore, instead of learning how one representation is mapped onto another (or, as in the case of WO2021/052896A1, how multiple representations are mapped onto one representation), the model learns the dynamic behavior of the examination region, i.e., the dynamics of how the states are passed through from one to another. The more states covered by the training data, the more accurately the model can learn the dynamics from one state to the subsequent states. Furthermore, a model trained in this way can be used to predict representations at states for which training data were unavailable, by multiple (iterative) use of the model to predict representations of further subsequent states. This extrapolation to states not taken into account during training is described in greater detail later in the description.

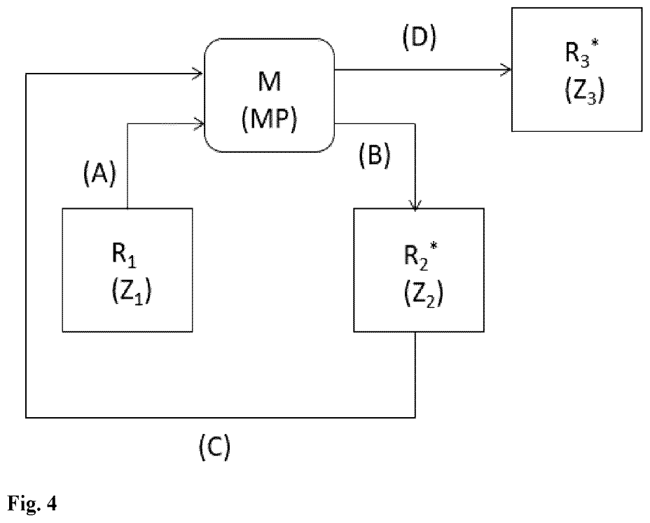

If the machine-learning model shown in FIG. 3 is trained, it can be used for prediction. This is shown in FIG. 4. In a first step (A), a first representation R₁ is fed to the trained machine-learning model M. The first representation R₁ represents the examination region of a new examination object in the first state Z₁. The term “new” can mean that no representations of the examination object were used when training the machine-learning model M. The model M generates in step (B) on the basis of the first representation R₁ and on the basis of the model parameters MP a second representation R₂*. The second representation R₂* represents the examination region of the examination object in the second state Z₂. In a third step (C), the generated second representation R₂* is fed to the machine-learning model M. The model M generates in step (D) on the basis of the second representation R₂* and on the basis of the model parameters MP a third representation R₃*. The third representation R₃* represents the examination region of the examination object in the third state Z₃.

The prediction method shown in FIG. 4 and based on the training method shown in FIG. 3 is a preferred embodiment of the present disclosure and comprises the steps of:
receiving a representation R₁ of an examination region, where the representation R₁ represents the examination region in a first state Z₁ of a sequence of states Z₁, Z₂, Z₃,
generating a representation R₂* of the examination region with the aid of a machine-learning model at least partly on the basis of the representation R₁, where the representation R₂* represents the examination region in the second state Z₂, where the second state Z₂ directly follows the first state Z₁ in the sequence of states,
generating a representation R₃* of the examination region with the aid of the machine-learning model at least partly on the basis of the representation R₂*, where the representation R₃* represents the examination region in the third state Z₃, where the third state Z₃ directly follows the second state Z₂ in the sequence of states,
outputting and/or storing and/or transmitting the representation R₃*,
   where the machine-learning model has been trained on the basis of training data, where the training data comprise a multiplicity of data sets, where each data set comprises a representation ^{T}R₁, a representation ^{T}R₂ and a representation ^{T}R₃, where the representation ^{T}R₁ represents the examination region in the first state Z₁ of the sequence of states, the representation ^{T}R₂ represents the examination region in the second state Z₂ of the sequence of states and the representation ^{T}R₃ represents the examination region in the third state Z₃ of the sequence of states, where the machine-learning model has been trained to generate at least partly on the basis of the representation ^{T}R₁ a representation ^{T}R₂* and to generate at least partly on the basis of the representation ^{T}R₂* a representation ^{T}R₃* for each examination object of a multiplicity of examination objects, where the training involves quantifying differences between the representations ^{T}R₂ and ^{T}R₂* and between the representations ^{T}R₃ and ^{T}R₃* and modifying model parameters in order to minimize the differences.

In general, the machine-learning model according to the invention may be trained to predict a representation of an examination region of an examination object in one state Zᵢ of a sequence of states Z₁ to Zₙ, where n is an integer greater than 2 and i is an index passing through the numbers from 2 to n.

The machine-learning model may be trained to generate, starting from a first representation ^{T}R₁ of the examination region in the state Z₁, a sequence of representations ^{T}R₁* to ^{T}Rₖ* of the examination region in the states Z₂ to Zₙ one after the other, where each representation of one state is generated at least partly on the basis of the respective previously generated representation of the directly preceding state.

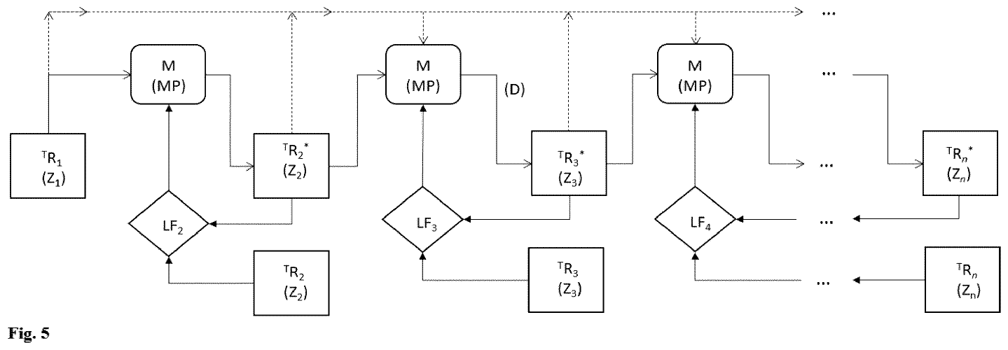

This shall be explained in greater detail using the example of FIG. 5. FIG. 5 can be understood as an extension of the scheme shown in FIG. 3 from three states to n states, where n is an integer greater than two. Preferably, the number n is in the range from 3 to 100.

FIG. 5 shows a machine-learning model M. The model M is specified three times, but it is always the same model. The machine-learning model M is trained to predict a number n−1 of representations one after the other. In the present example, n is an integer greater than 3. The model is fed a first representation ^{T}R₁ as input data. The first representation ^{T}R₁ represents an examination region of an examination object in a first state Z₁. The machine-learning model M is configured to generate at least partly on the basis of the first representation ^{T}R₁ and on the basis of model parameters MP an output ^{T}R₂*. The output ^{T}R₂* is a predicted second representation representing the examination region in a second state Z₂. The model is further configured to generate at least partly on the basis of the output ^{T}R₂* and model parameters MP an output ^{T}R₃*. The output ^{T}R₃* is a predicted third representation representing the examination region in a third state Z₃. The model is further configured to generate at least partly on the basis of the output ^{T}R₃* and model parameters MP a further output showing a predicted representation of the examination region in one state following the state Z₃. This scheme is continued up to an output ^{T}Rₙ*. The output ^{T}Rₙ* is a predicted n-th representation representing the examination region in an n-th state Zₙ.

The states Zᵢ (where i=1 to n) form a sequence of states (Z₁→Z₂→Z₃→ . . . →Zₙ).

Present in the case of the example shown in FIG. 5 are not only the first representation ^{T}R₁, but also further (actual, measurement-generated) representations ^{T}Rⱼ (where j=2 to n) of the examination region in the states Z (where j=2 to n) that can be used as ground truth data for training of the machine-learning model. For instance, the difference between an output ^{T}Rⱼ* (which is a predicted j-th representation of the examination region in the state Zⱼ) and the representation ^{T}Rⱼ may be quantified using a loss function LFⱼ: a loss function LF₂ may quantify the difference between the representation ^{T}R₂ and the generated representation ^{T}R_{2*}, a loss function LF₃ may quantify the difference between the representation ^{T}R₃ and the generated representation ^{T}R_{3*}, and so on.

In an end-to-end training, a total loss function LF taking into account all individual differences may be used; for example, this may be the sum of the individual differences:

$$LF = {LF}_{2} + {LF}_{3} + … + {LF}_{n} = {\sum }_{j = 2}^{n} {LF}_{j}$$

Alternatively, the sum may be a weighted sum:

$$LF = {w}_{2} \cdot {LF}_{2} + {w}_{3} \cdot {LF}_{3} + … + {w}_{n} \cdot {LF}_{n} = {\sum }_{j = 2}^{n} {w}_{j} \cdot {LF}_{j}$$

where the weight factors wⱼ may assume, for example, values from 0 to 1.

The advantage of weighting is that, when generating a representation ^{T}Rₙ representing the examination region in one state Zₙ, more weight or less weight is given to one or more (generated) representations representing one state before the state Zₙ than to other representations. For example, it is possible that the weight factors w increase in the order w₂, w₃, . . . , wₙ and thus more weight is given to representations associated with states closer to the state Zₙ. The increase may be logarithmic, linear, square, cubic or exponential, or it may be some other increase. It is also conceivable to give less weight to later states, in that the weight factors decrease in the order w₂, w₃, . . . , wₙ and thus more weight is given to representations associated with states closer to the initial state Z₁. In the case too of such a decrease, it may be logarithmic, linear, square, cubic or exponential, or it may be some other decrease.

It is also conceivable that the training data comprise incomplete data sets. This means that some data sets of examination objects do not comprise all representations ^{T}R₁ to ^{T}Rₙ. As explained later in the description, incomplete data sets may also be used for training of the machine-learning model. If a representation ^{T}Rₚ is missing, the weight factor wₚ which is to weight the differences between the representation ^{T}Rₚ and the generated representation Rₚ* may be set to zero, where p is an integer which may assume the values of 2 to n.

It is also conceivable that training involves considering random numbers 1≤j<k≤n and the associated partial sequences of states Zⱼ, Zⱼ₊₁, . . . , Zₖ₋₁, Zₖ. The learning problem described above may then be solved on the basis of these partial sequences (which optionally vary from time to time). For example, it is conceivable that a random initial state Zⱼ (1≤j≤n−2) is determined and the model is to always synthesize the representations of the subsequent two states Zⱼ+1, Zⱼ+2 on the basis thereof.

In FIG. 5, it is also shown schematically that a representation ^{T}Rⱼ* representing the examination region in the state Zⱼ may be generated by using not only the representation ^{T}Rⱼ₋₁* representing the examination region in the preceding state Zⱼ₋₁, but also further (generated) representations ^{T}Rⱼ₋₂* and/or ^{T}Rⱼ₋₃* and/or . . . to ^{T}R₁ representing the examination region in further preceding states Zⱼ₋₂, Zⱼ₋₃ . . . to Z₁, where j is an integer which may assume the values of 2 to n. This is expressed by the dashed arrows. For instance, the representation ^{T}R₃* may be generated by feeding not only the representation ^{T}R_{2*}, but also the representation ^{T}R₁, to the machine-learning model M. The representation ^{T}R₄* may be generated by feeding not only the representation ^{T}R_{3*}, but also the representation ^{T}R₂* and/or the representation ^{T}R₁, to the machine-learning model M.

To generate representations, additional information may also be incorporated into the machine-learning model, such as information about the state which the examination region is in, the representation of which is used to generate a representation in a subsequent state. In other words, the representation ^{T}R₂* may be generated by using not only the representation ^{T}R₁, but also information about the state which the examination region represented by the representation ^{T}R₁ is in. The representation ^{T}R₃* too may be generated on the basis of the representation ^{T}R₂* and information about the second state Z₂.

If, during training and/or use of the trained machine-learning model for prediction, information is provided about the respective state of a representation, the machine-learning model will “know” where in the sequence of states it is respectively in.

For example, one state may be represented by a number or a vector. For example, the states may be numbered consecutively, such that the first state is represented by the number 1, the second state by the number 2, and so on.

Instead of or in addition to information about the respective state, a time that can be assigned to the respective state may also be used in the generation of a representation. For example, the first state in the sequence of states may be assigned the time point t=0. Time t=0 may be input into the machine-learning model together with a first representation in order to predict a second representation. The second state may be assigned a number of seconds or minutes, or some other unit of a time difference, that indicates how much time has elapsed between the first state and the second state. This time difference, together with the predicted second representation (and optionally the first representation), may be fed to the machine-learning model in order to predict a third representation. The third state may likewise be assigned a time difference that indicates how much time has elapsed between the first state and the third state. This time difference, together with the predicted third representation (and optionally the first representation and/or the predicted second representation), may be fed to the machine-learning model in order to predict the third representation. And so on.

The machine-learning model according to the invention may also be understood to mean a transformation which may be applied to a representation of an examination region in one state of a sequence of states in order to predict a representation of the examination region in a subsequent state of the sequence of states. The transformation may be applied singly (once) in order to predict a representation of the examination region in the immediately subsequent state, or it may be applied multiply (multiple times, iteratively) in order to predict a representation of the examination region in one state further downstream in the sequence of states.

If there is a sequence of n states Z₁ to Zₙ, and if at each state Zᵢ there is a representation Rᵢ representing the examination region in the state Zᵢ, the machine-learning model M may be applied q times starting from the representation R₁ in order to generate a predicted representation R_{1+q}* representing the examination region in the state Z_{1+q}, where q may assume the values of 1 to n−1:

$${M}^{q} ( {R}_{1} ) = {R}_{1 + q}^{*}$$

$$q = 1 : M ( {R}_{1} ) = {R}_{2}^{*}$$

$$q = 2 : M ( {R}_{2}^{*} ) = M ( M ( {R}_{1} ) ) = {M}^{2} ( {R}_{1} ) = {R}_{3}^{*}$$

$$q = 3 : M ( {R}_{3}^{*} ) = M ( M ( {R}_{2}^{*} ) ) = M ( M ( M ( {R}_{1} ) ) ) = {M}^{3} ( {R}_{1} ) = {R}_{4}^{*}$$

$$…$$

$$q = n - 1 : M ( {R}_{n - 1}^{*} ) = M ( M ( {R}_{n - 2}^{*} ) ) = … = {M}^{n - 1} ( {R}_{1} ) = {R}_{n}^{*}$$

This also applies analogously to the training method, in which the q-times transformation M may be described by the following formula:

M^{q}(^{T}R₁)=^{T}R_{1+q}*

A loss function quantifying all differences between generated representations ^{T}R_{q}* and actual (measurement-generated) representations ^{T}R_{q} may be expressed, for example, by the following formula:

$$LV = {w}_{2} \cdot d ( M ( {}^{T} {R}_{1} ) , {}^{T} {R}_{2} ) + {w}_{3} \cdot d ( {M}^{2} ( {}^{T} {R}_{1} ) , {}^{T} {R}_{3} ) + … + {w}_{n} \cdot d ( {M}^{n - 1} ( {}^{T} {R}_{1} ) , {}^{T} {R}_{n} )$$

Here, LV is the loss value produced for a data set comprising the (actual, measurement-generated) representations ^{T}R₁, ^{T}R₂, . . . , ^{T}Rₙ. d is a loss function quantifying the differences between a predicted representation M(^{T}R_{q-1}) and the representation ^{T}R_{q}. As already described earlier in the description, this may be, for example, one of the following loss functions: L1 loss, L2 loss, Lp loss, structural similarity index measure (SSIM), VGG loss, perceptual loss or a combination thereof.

w₂, w₃, . . . , wₙ are weight factors that have likewise already been described earlier in the description.

n indicates the number of states.

It is also conceivable that the loss value is the maximum difference calculated for a data set:

LV=max(w₂·d(M(^{T}R₁),^{T}R₂);w₃·d(M²(^{T}R₁),^{T}R₃); . . . ;wₙ·d(Mⁿ⁻¹(^{T}R₁),^{T}Rₙ))

and in this formula too, the weight factors can also be used to give a different weight to the individual differences.

As already indicated earlier in the description, it should be noted that training of the machine-learning model does not require that each data set of the training data contain representations of all states to be learnt by the model. Two representations per data set are sufficient in principle. This shall be explained using an example. Assume that the machine-learning model is to be trained to predict representations of an examination region in the states of a sequence of 6 states Z₁ to Z₆. Assume that training data comprising 10 data sets of 10 examination objects are sufficient for training the machine-learning model. Each data set comprises representations of the examination region in different states, for example:
Data set 1: ^{T}R₁, ^{T}R₃, ^{T}R₄, ^{T}R₅, ^{T}R₆
Data set 2: ^{T}R₁, ^{T}R₂, ^{T}R₄, ^{T}R₆
Data set 3: ^{T}R₁, ^{T}R₂, ^{T}R₃, ^{T}R₄, ^{T}R₅
Data set 4: ^{T}R₁, ^{T}R₂, ^{T}R₃, ^{T}R₅, ^{T}R₆
Data set 5: ^{T}R₂, ^{T}R₃, ^{T}R₅, ^{T}R₆
Data set 6: ^{T}R₂, ^{T}R₃, ^{T}R₄, ^{T}R₅, ^{T}R₆
Data set 7: ^{T}R₂, ^{T}R₃, ^{T}R₅, ^{T}R₆
Data set 8: ^{T}R₃, ^{T}R₅, ^{T}R₆
Data set 9: ^{T}R₃, ^{T}R₄, ^{T}R₅, ^{T}R₆
Data set 10: ^{T}R₃, ^{T}R₄, ^{T}R₆

In the example, there is no single data set that is “complete”, i.e., no data set comprising all possible representations ^{T}R₁, ^{T}R₂, ^{T}R₃, ^{T}R₄, ^{T}R₅ and ^{T}R₆. Nevertheless, it is possible to train the machine-learning model on the basis of such training data to predict a representation of each state. This is another advantage of the present invention over the “direct training” described above.

Once the machine-learning model is trained, the model can be fed new (i.e., not used in training) representations of an examination region of a (new) examination object in one state of a sequence of states, and the model can predict (generate) one or more representations of the examination region in a subsequent state or in multiple subsequent states of the sequence of states.

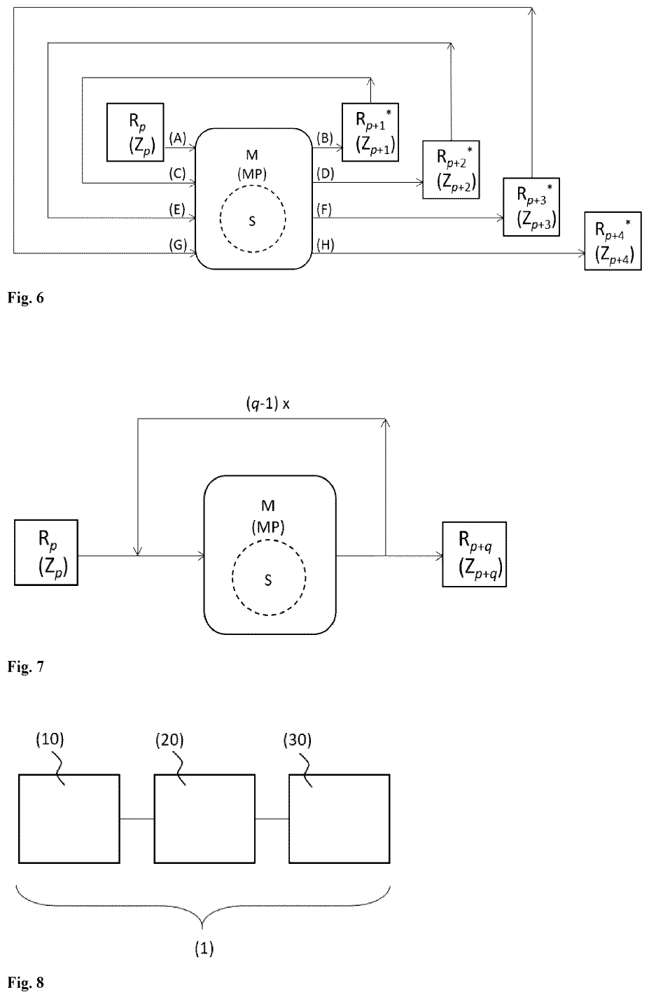

This is depicted by way of example and in schematic form in FIG. 6. In the example shown in FIG. 6, what is generated starting from a representation Rₚ representing the examination region in the state Zₚ is a sequence of representations Rₚ₊₁*, Rₚ₊₂*, Rₚ₊₃* and Rₚ₊₄* representing the examination region in the states Zₚ₊₁, Zₚ₊₂, Zₚ₊₃ and Zₚ₊₄. The states Zₚ₊₁, Zₚ₊₂, Zₚ₊₃ and Zₚ₊₄ form a sequence of states: Zₚ₊₁→Zₚ₊₂→Zₚ₊₃→Zₚ₊₄.

In step (A), the machine-learning model M is fed the representation Rₚ. Besides the representation Rₚ, the machine-learning model M may also be fed information about the state Zₚ and/or additional/other information, as described in this description.

The machine-learning model M generates in step (B) on the basis of the fed input data the representation Rₚ₊₁* representing the examination region in the state Zₚ₊₁.

In step (C), the machine-learning model M is fed the previously generated representation Rₚ₊₁*. Besides the representation Rₚ₊₁*, the machine-learning model M may also be fed information about the state Zₚ₊₁ and/or additional information. Furthermore, the machine-learning model M may also be fed the representation Rₚ and/or information about the state Zₚ.

Preferably, the machine-learning model M comprises a memory S which stores input data (and preferably also output data), so that one-time entered input data and/or generated output data need not be received and/or entered again, but are already available to the machine-learning model. This applies not only to the utilization of the trained machine-learning model for prediction as described in this example, but also to the training of the machine-learning model according to the invention.

The machine-learning model M generates in step (D) on the basis of the fed input data the representation Rₚ₊₂* representing the examination region in the state Zₚ₊₂.

In step (E), the machine-learning model M is fed the previously generated representation Rₚ₊₂*. Besides the representation Rₚ₊₂*, the machine-learning model M may also be fed information about the state Zₚ₊₂ and/or additional information. Furthermore, the machine-learning model M may also be fed the representation Rₚ and/or the representation Rₚ₊₁* and/or information about the state Zₚ and/or the state Zₚ₊₁.

The machine-learning model M generates in step (F) on the basis of the fed input data the representation Rₚ₊₃* representing the examination region in the state Zₚ₊₃.

In step (G), the machine-learning model M is fed the previously generated representation Rₚ₊₃*. Besides the representation Rₚ₊₃*, the machine-learning model M may also be fed information about the state Zₚ₊₃ and/or additional information. Furthermore, the machine-learning model M may also be fed the representation Rₚ and/or the representation Rₚ₊₁* and/or the representation Rₚ₊₂* and/or information about the state Zₚ and/or the state Zₚ₊₁ and/or the state Zₚ₊₂.

The machine-learning model M generates in step (H) on the basis of the fed input data the representation Rₚ₊₄* representing the examination region in the state Zₚ₊₄.

The generated representations Rₚ₊₁*, Rₚ₊₂*, Rₚ₊₃* and/or Rₚ₊₄* may be output (e.g., displayed on a monitor and/or printed by a printer) and/or stored in a data memory and/or transmitted to a (separate) computer system.

If the machine-learning model has been trained to generate, starting from a first representation ^{T}R₁ representing the examination region in a first state Z₁, a sequence of representations ^{T}R_{2*}, . . . , ^{T}Rₙ*, where each representation ^{T}Rᵢ represents the examination region in one state Zⱼ and where j is an index passing through the numbers from 2 to n, then such a model can be fed a new representation Rₚ representing the examination region in the state Zₚ and the machine-learning model can generate the representations Rₚ₊₁*, Rₚ₊₂*, . . . , Rₙ*, where p is a number which may assume the values of 2 to n.

This means that the trained model does not necessarily have to be fed a new representation R₁ representing the examination region in the first state Z₁. Furthermore, the trained machine-learning model does not necessarily have to generate all representations from Rₚ₊₁* to Rₙ*. Instead, it is possible to “drop in” and “drop out” anywhere in the sequence of states Z₁ to Zₙ and thus generate on the basis of a representation Rₚ one or more representations representing the examination region at one or more subsequent states.

It is even possible to generate representations representing states that were not addressed at all in the training. Thus, instead of stopping at the representation Rₙ*, predictions may also be made of the representations Rₙ₊₁*, Rₙ₊₂* and so on. The trained machine-learning model may thus be used to continue the learned dynamics and to calculate representations of states that were never generated by measurement. In this respect, the trained machine-learning model may be used to extrapolate to new states.

Furthermore, the predictions are not restricted to subsequent states. It is also possible to predict representations of the examination region in a preceding state of the sequence of states. Firstly, as already described earlier in the description, the machine-learning model may fundamentally be trained in both directions: in the direction of subsequent states and in the direction of preceding states. Secondly, the machine-learning model performs on an entered representation a transformation that in principle can also be reversed. Analysis of the mathematical functions of the model that transform an input representation into an output representation makes it possible to determine inverse functions that reverse the process and change the previous output representation back into the previous input representation.

The inverse functions may then be used to predict representations of preceding states, even if the model was trained to predict representations of subsequent states, and vice versa.

FIG. 7 shows an extension of the scheme shown in FIG. 6. Whereas FIG. 6 shows how, on the basis of a representation Rₚ representing the examination region in the state Zₚ, the representations Rₚ₊₁*, Rₚ₊₂*, Rₚ₊₃* and Rₚ₊₄* representing the examination region in the states Zₚ₊₁, Zₚ₊₂, Zₚ₊₃ and Zₚ₊₄ are generated one after the other, FIG. 7 shows how, on the basis of the representation Rₚ representing the examination region in the state Zₚ, the representations Rₚ₊₁* to R_{p+q}* are generated one after the other, where q is an integer greater than 1. In FIG. 7, the iterative character of the machine-learning model M is expressed particularly clearly. The machine-learning model M is, starting from the representation Rₚ, applied q times, and the output data are returned (q−1) times back into the machine-learning model M ((q−1) x).

It should be noted that the (trained or untrained) machine-learning model does not have to be applied to a complete radiological image (e.g., an MRI image or a CT scan or the like). It is possible to apply the machine-learning model to only part of a radiological image. It is possible, for example, to segment a radiological image first in order to identify/select a region of interest. The model may then, for example, be applied solely to the region of interest.

Furthermore, the application of the machine-learning model may comprise one or more preprocessing and/or postprocessing steps. For example, it is conceivable to first subject a received representation of the examination region to one or more transformations, such as motion correction, color space conversion, normalization, segmentation, Fourier transform (e.g., for conversion from an image-space representation into a frequency-space representation), inverse Fourier transform (e.g., for conversion from a frequency-space representation into an image-space representation) and/or the like. In a further step, the transformed representation may be fed to the machine-learning model, which then passes through a series of iterations (cycles) (as shown schematically in FIG. 7) in order to generate, starting from the transformed representation, a series of further (subsequent) representations of the examination region in a series of further (subsequent) states.

The machine-learning model according to the invention may, for example, be an artificial neural network or include such a network.

An artificial neural network comprises at least three layers of processing elements: a first layer with input neurons (nodes), an N-th layer with at least one output neuron (nodes) and N−2 inner layers, where N is a natural number and greater than 2.

The input neurons serve to receive the representations. There may for example be one input neuron for each pixel or voxel of a representation when the representation is a real-space depiction in the form of a raster graphic, or be one input neuron for each frequency present in the representation when the representation is a frequency-space depiction. There may be additional input neurons for additional input values (e.g., information about the examination region, information about the examination object, information about the conditions when the representation was generated, information about the state represented by the representation, and/or information about the time at which or period of time in which the representation was generated).

The output neurons may serve to output a generated representation representing the examination region in a subsequent state.

The processing elements of the layers between the input neurons and the output neurons are connected to one another in a predetermined pattern with predetermined connection weights.

Preferably, the artificial neural network is a so-called convolutional neural network (CNN for short) or includes such a network.

A CNN normally consists essentially of an alternately repeating array of filters (convolutional layer) and aggregation layers (pooling layer) terminating in one or more layers of fully connected neurons (dense/fully connected layer).

The artificial neural network may be trained, for example, by means of a backpropagation method. The goal for the network is to predict the dynamics of the examination region from one state via at least one intermediate state to an end state as reliably as possible. The quality of prediction is described by a loss function. The goal is to minimize the loss function. In the case of the backpropagation method, an artificial neural network is taught by the alteration of the connection weights.

In the trained state, the connection weights between the processing elements contain information regarding the dynamics of state changes that may be used in order to predict, on the basis of a first representation representing the examination region in a first state, one or more representations representing the examination region in one or more subsequent states.

A cross-validation method may be used in order to divide the data into training and validation data sets. The training data set is used in the backpropagation training of network weights. The validation data set is used in order to check the accuracy of prediction with which the trained network can be applied to unknown data.

The artificial neural network may have an autoencoder architecture; for example, the artificial neural network may have an architecture such as U-Net (see for example O. Ronneberger et al.: U-net: Convolutional networks for biomedical image segmentation, International Conference on Medical image computing and computer-assisted intervention, pages 234-241, Springer, 2015, https://doi.org/10.1007/978-3-319-24574-4_28).

The artificial neural network may be a generative adversarial network (GAN) (see for example M.-Y. Liu et al.: Generative Adversarial Networks for Image and Video Synthesis: Algorithms and Applications, arXiv: 2008.02793; J. Henry et al.: Pix2Pix GAN for Image-to-Image Translation, DOI: 10.13140/RG.2.2.32286.66887).

The artificial neural network may be a recurrent neural network or include such a network. Recurrent or feedback neural networks refer to neural networks that, in contrast to feed-forward networks, are distinguished by connections between neurons of one layer and neurons of the same layer or a preceding layer. The artificial neural network may for example include a long short-term memory (LSTM) (see for example Y. Gao et al.: Fully convolutional structured LSTM networks for joint 4D medical image segmentation, DOI: 10.1109/ISBI.2018.8363764).

The artificial neural network may be a transformer network (see for example D. Karimi et al.: Convolution-Free Medical Image Segmentation using Transformers, arXiv:2102.13645 [eess.IV]).

FIG. 8 shows by way of example and in schematic form a computer system according to the present disclosure.

A “computer system” is an electronic data processing system that processes data by means of programmable calculation rules. Such a system typically comprises a “computer”, which is the unit that includes a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (1) shown in FIG. 8 comprises an input unit (10), a control and calculation unit (20) and an output unit (30).

The control and calculation unit (20) serves for control of the computer system (1), for coordination of the data flows between the units of the computer system (1), and for the performance of calculations.

The control and calculation unit (20) is configured
to receive via the input unit (10) a representation Rₚ representing an examination region of an examination object in one state Zₚ of a sequence of states Z₁ to Zₙ, where n is an integer greater than two, and p is an integer less than n,
to feed the received representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region in the state Z₁ and each generated representation ^{T}Rⱼ* represents the examination region in the state Zⱼ, where j is an index passing through the numbers from 2 to n, where the representation ^{T}R₂* is generated at least partly on the basis of the representation ^{T}R₁ and each further representation ^{T}Rₖ is generated at least partly on the basis of the generated representation ^{T}Rₖ₋₁*, where k is an index passing through the numbers from 3 to n,
to receive from the machine-learning model one or more representations of the examination region, where each of the one or more representations represents the examination region in one state that follows the state Zₚ,
to cause the output unit (30) to output the one or more representations received from the machine-learning model and/or to store them and/or to transmit them to a separate computer system.

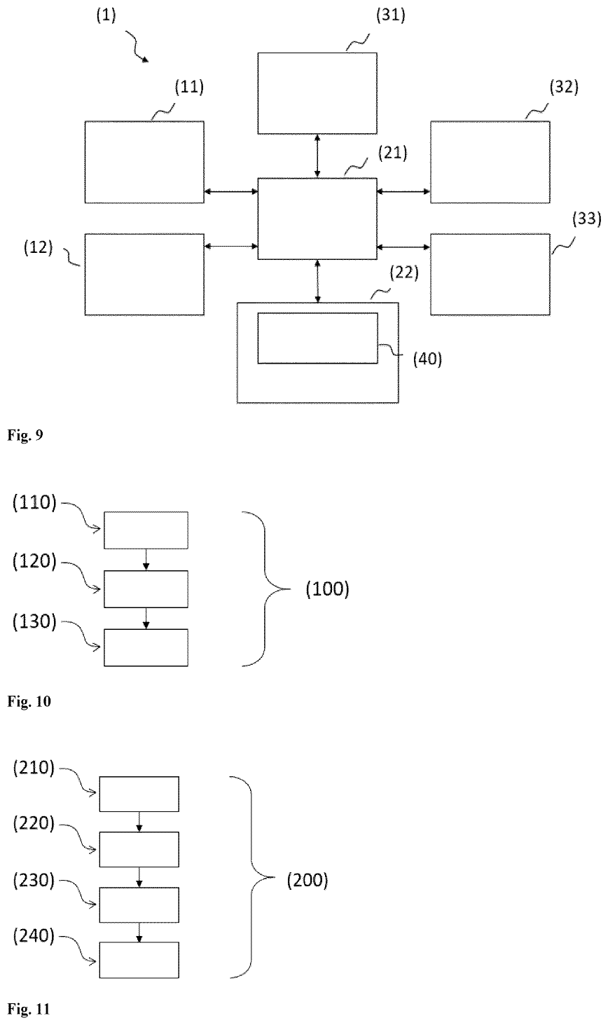

FIG. 9 shows by way of example and in schematic form a further embodiment of the computer system according to the invention.

The computer system (1) comprises a processing unit (21) connected to a memory (22). The processing unit (21) and the memory (22) form a control and calculation unit, as shown in FIG. 8.

The processing unit (21) may comprise one or more processors alone or in combination with one or more memories. The processing unit (21) may be customary computer hardware that is able to process information such as digital images (e.g., representations of the examination region), computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of same or of a different computer system.

The memory (22) may be customary computer hardware that is able to store information such as digital images (for example representations of the examination region), data, computer programs and/or other digital information either temporarily and/or permanently. The memory (22) may comprise a volatile and/or nonvolatile memory and may be nonremovable or removable. Examples of suitable memories are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (21) may be connected not just to the memory (22), but also to one or more interfaces (11, 12, 31, 32, 33) in order to display, transmit and/or receive information. The interfaces may comprise one or more communication interfaces (32, 33) and/or one or more user interfaces (11, 12, 31). The one or more communication interfaces (32, 33) may be configured to send and/or receive information, for example to and/or from an MRI scanner, a CT scanner, an ultrasound camera, other computer systems, networks, data memories or the like. The one or more communication interfaces (32, 33) may be configured to transmit and/or receive information via physical (wired) and/or wireless communication connections. The one or more communication interfaces (32, 33) may comprise one or more interfaces for connection to a network, for example using technologies such as mobile telephone, Wi-Fi, satellite, cable, DSL, optical fiber and/or the like. In some examples, the one or more communication interfaces (32, 33) may comprise one or more close-range communication interfaces configured to connect devices having close-range communication technologies such as NFC, RFID, Bluetooth, Bluetooth LE, ZigBee, infrared (e.g. IrDA) or the like.

The user interfaces (11, 12, 31) may comprise a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image or video recording device (for example a camera), a keyboard or a keypad, a joystick, a touch-sensitive surface (separate from a touchscreen or integrated therein) or the like. In some examples, the user interfaces (11, 12, 31) may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces (11, 12, 31) may furthermore comprise one or more interfaces for communication with peripherals such as printers and the like.

One or more computer programs (40) may be stored in the memory (22) and executed by the processing unit (21), which is thereby programmed to perform the functions described in this description. The retrieving, loading and execution of instructions of the computer program (40) may take place sequentially, such that an instruction is respectively retrieved, loaded and executed. However, the retrieving, loading and/or execution may also take place in parallel.

The machine-learning model of the present disclosure may also be stored in the memory (22).

The computer system of the present disclosure may be designed as a laptop, notebook, netbook and/or tablet PC; it may also be a component of an MRI scanner, a CT scanner or an ultrasound diagnostic device.

FIG. 10 shows schematically in the form of a flow chart one embodiment of a method for training a machine-learning model.

The method (100) comprises:
(110) receiving training data,
   where the training data comprise representations ^{T}R₁ to ^{T}Rₙ of an examination region for a multiplicity of examination objects, where n is an integer greater than two,
   where each representation ^{T}Rᵢ represents the examination region in one state Zᵢ of a sequence of states Z₁ to Zₙ, where i is an index passing through the integers from 1 to n,
(120) training the machine-learning model,
   where the model is trained to generate, starting from the representation ^{T}R₁, representations ^{T}R₂* to ^{T}Rₙ* one after the other,
   where the representation ^{T}R₂* is generated at least partly on the basis of the representation ^{T}R₁ and each further representation ^{T}Rₖ is generated at least partly on the basis of the representation ^{T}Rₖ₋₁*, where k is an index passing through the numbers from 3 to n,
   where the representation ^{T}Rₖ represents the examination region in the state Zₖ and the representation ^{T}Rₖ₋₁* represents the examination region in the state Zₖ₋₁, and the state Zₖ₋₁ directly precedes the state Zₖ,
   where differences between the generated representations ^{T}Rⱼ* and the representations ^{T}Rⱼ are quantified by means of a loss function, where j is an index passing through the numbers from 2 to n,
   where the differences are minimized by modifying model parameters of the machine-learning model,
(130) storing the trained machine-learning model and/or utilizing the machine-learning model for prediction.

FIG. 11 shows schematically in the form of a flow chart one embodiment of a computer-implemented method for generating a representation in one state.

The method (200) comprises:
(210) receiving a representation Rₚ of an examination region of an examination object,
   where the representation R₂ represents the examination region in one state Zₚ of a sequence of states Z₁ to Zₙ, where p is an integer less than n, where n is an integer greater than two,
(220) feeding the representation Rₚ to a trained machine-learning model,
   where the machine-learning model has been trained according to the method (100),
(230) receiving one or more representations R_{p+q}* of the examination region from the machine-learning model,
   where each of the one or more representations R_{p+q}* represents the examination region in one state Z_{p+q},
   where q is an index passing through the numbers from 1 to m,
   where m is an integer less than n−1 or equal to n−1 or greater than n−1,
(240) outputting and/or storing and/or transmitting the one or more representations R_{p+q}*.

1. A computer-implemented method for training a machine-learning model, wherein the method comprises:
receiving training data:
wherein the training data comprise representations ^{T}R₁ to ^{T}Rₙ of an examination region for a multiplicity of examination objects, where n is an integer greater than two,
wherein each representation ^{T}Rᵢ represents the examination region in one state Zᵢ of a sequence of states Z₁ to Zₙ, where i is an index passing through the integers from 1 to n, and
wherein each examination object is preferably a human and the examination region is preferably part of the human;
training the machine-learning model;
wherein the model is trained to generate, starting from the representation ^{T}R₁, representations ^{T}R₂ to ^{T}Rₙ* one after the other for each examination object of the multiplicity of examination objects,
wherein the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n, and
wherein the representation ^{T}Rₖ represents the examination region in the state Zₖ and the representation ^{T}Rₖ₋₁* represents the examination region in the state Zₖ₋₁, and the state Zₖ₋₁ directly precedes the state Zₖ; and
storing the trained machine-learning model and/or utilizing the machine-learning model for prediction.

2. The method of claim 1, further comprising:
for each generated representation ^{T}Rⱼ*: calculating a loss value for a pair composed of the received representation ^{T}Rⱼ and the generated representation ^{T}Rⱼ* with the aid of a loss function, where j is an index passing through the numbers from 2 to n;
calculating a total loss value with the aid of a total loss function, where the total loss function is a function of the loss values; and
minimizing the total loss value by modifying parameters of the machine-learning model.

3. The method of claim 2, wherein the total loss function has the following formula:
LV=Σⱼ₌₂ⁿwⱼ·LFⱼ
wherein LV is the total loss value, where LFⱼ are the loss functions for calculating the loss values for the differences between the received representation ^{T}Rⱼ and the generated representation ^{T}Rⱼ*, and wⱼ are weight factors.

4. A computer-implemented method for predicting one or more representations of an examination region of an examination object, comprising:
receiving a representation Rₚ of the examination region;
wherein the representation Rₚ represents the examination region in one state Zₚ of a sequence of states Z₁ to Zₙ, where p is an integer less than n, where n is an integer greater than two, and
wherein the examination object is preferably a human and the examination region is preferably part of the human;
feeding the representation Rₚ to a trained machine-learning model:
wherein the trained machine-learning model has been trained on the basis of training data to generate, starting from a first representation ^{T}R₁, a number n−1 of representations ^{T}R₂* to ^{T}Rₙ* one after the other,
wherein the first representation ^{T}R₁ represents the examination region in the first state Z₁ and each generated representation ^{T}Rⱼ* represents the examination region in the state Zⱼ, where j is an index passing through the numbers from 2 to n,
wherein the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n, and
wherein the training data are the result of a radiological examination;
receiving one or more representations R_{p+q} of the examination region from the machine-learning model;
wherein each of the one or more representations R_{p+q}* represents the examination region in the state Z_{p+q}, where q is an index passing through the numbers from 1 to m, where m is an integer less than n−1 or equal to n−1 or greater than n−1; and
outputting and/or storing and/or transmitting the one or more representations R_{p+q}*.

5. The method of claim 4, wherein the method comprises:
receiving the representation Rₚ of the examination region, where the representation Rₚ represents the examination region in the state Zₚ from the sequence of states Z₁ to Zₙ, where p is an integer less than n,
feeding the representation Rₚ to the trained machine-learning model,
receiving the one or more representations R_{p+q}* of the examination region from the trained machine-learning model, where each of the one or more representations R_{p+q}* represents the examination region in the state Z_{p+q}*, where q is an index passing through the numbers from 1 to m, where m is an integer less than n−1 or equal to n−1 or greater than n−1, and
outputting and/or storing and/or transmitting the one or more representations R_{p+q}*.

6. The method of claim 4, as claimed in any of claims 1 to 5, wherein each representation R_{1+q}*is calculated according to the following formula:
M^{q}(R₁)=R_{1+q}*
wherein M represents a transformation which is applied by the machine-learning model to input data of the machine-learning model, where M^{q} means the q-times application of the transformation, where in a first step the transformation is applied to a first representation R₁, in a second step the transformation is applied to the result of the application in the first step in that the result is passed back into the machine-learning model and the procedure is repeated with each further result of a transformation until the transformation has been applied a total of q times, where q is an integer which may assume the values of 1 to m, where m is an integer less than n−1 or equal to n−1 or greater than n−1.

7. The method of claim 4, wherein the sequence of states comprises one or more of the following states:
a first state of the examination region at a first time point before the administration of a contrast agent,
a second state of the examination region at a second time point, after the administration of the contrast agent,
a third state of the examination region at a third time point, after the administration of the contrast agent,
a fourth state of the examination region at a fourth time point, after the administration of the contrast agent, and
a fifth state of the examination region at a fifth time point, after the administration of the contrast agent,
wherein the first time point, the second time point, the third time point, the fourth time point, and the fifth time point form a chronological sequence.

8. The method of claim 4, wherein the examination region is the human liver or is part of the human liver.

9. The method of claim 8, wherein the sequence of states comprises one or more of the following states:
the liver or part of the liver before the administration of a hepatobiliary contrast agent,
the liver or part of the liver during the arterial phase after the administration of the hepatobiliary contrast agent,
the liver or part of the liver during the portal venous phase after the administration of the hepatobiliary contrast agent,
the liver or part of the liver during the transitional phase after the administration of the hepatobiliary contrast agent, and
the liver or part of the liver during the hepatobiliary phase after the administration of the hepatobiliary contrast agent.

10. The method of claim 4, wherein each representation R_{p+q}* and ^{T}R_{p+q}* generated by the machine-learning model is returned to the trained machine-learning model in order to generate a representation R_{p+q+1}* and/or ^{T}R_{p+q+1}*.

11. The method of claim 4, wherein m is in the range from n to n+2.

12. The method of claim 4, wherein the received representation Rₚ is a CT image, MRI image or ultrasound image.

13. (canceled)

14. A computer program product comprising a computer program that can be loaded into a working memory of a computer system, where it causes the computer system to execute the following-steps:
receiving a representation Rₚ of an examination region of an examination object, where the representation Rₚ represents the examination region in one state Zₚ of a sequence of states Z₁ to Zₙ, where p is an integer less than n, where n is an integer greater than two, where the examination object is preferably a human and the examination region is preferably part of the human,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region in the first state Z₁ and each generated representation ^{T}Rⱼ* represents the examination region in the state Zⱼ, where j is an index passing through the numbers from 2 to n, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n,
receiving one or more representations R_{p+q}* of the examination region from the machine-learning model, where each of the one or more representations R_{p+q}* represents the examination region in the state Z_{p+q}, where q is an index passing through the numbers from 1 to m, where m is an integer less than n−1 or equal to n−1 or greater than n−1, and
outputting and/or storing and/or transmitting the one or more representations R_{p+q}*.

15-16. (canceled)

17. A kit comprising a contrast agent and the computer program product of claim 14.

18. The method of claim 5, where the trained machine-learning model has been trained, according to a computer-implemented method comprising:
receiving training data:
wherein the training data comprise representations ^{T}R₁ to ^{T}Rₙ of an examination region for a multiplicity of examination objects, where n is an integer greater than two,
wherein each representation ^{T}Rᵢ represents the examination region in one state Zᵢ of a sequence of states Z₁ to Zₙ, where i is an index passing through the integers from 1 to n, and
wherein each examination object is preferably a human and the examination region is preferably part of the human;
training the machine-learning model:
wherein the model is trained to generate, starting from the representation ^{T}R₁, representations ^{T}R₂ to ^{T}Rₙ* one after the other for each examination object of the multiplicity of examination objects,
wherein the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through the numbers from 3 to n, and
wherein the representation ^{T}Rₖ* represents the examination region in the state Zₖ and the representation ^{T}Rₖ₋₁* represents the examination region in the state Zₖ₋₁, and the state Zₖ₋₁ directly precedes the state Zₖ; and
storing the trained machine-learning model and/or utilizing the machine-learning model for prediction.

19. The method of claim 1, wherein each representation ^{T}R_{1+q}* is calculated according to the following formula:
M^{q}(^{T}R₁)=^{T}R_{1+q}*
wherein M represents a transformation which is applied by the machine-learning model to input data of the machine-learning model, where M^{q} means the q-times application of the transformation, where in a first step the transformation is applied to a first representation ^{T}R₁, in a second step the transformation is applied to the result of the application in the first step in that the result is passed back into the machine-learning model and the procedure is repeated with each further result of a transformation until the transformation has been applied a total of q times, where q is an integer which may assume the values of 1 to m, where m is an integer less than n−1 or equal to n−1 or greater than n−1.

20. The method of claim 1, wherein the sequence of states comprises one or more of the following states:
a first state of the examination region at a first time point before the administration of a contrast agent,
a second state of the examination region at a second time point, after the administration of the contrast agent,
a third state of the examination region at a third time point, after the administration of the contrast agent,
a fourth state of the examination region at a fourth time point, after the administration of the contrast agent, and
a fifth state of the examination region at a fifth time point, after the administration of the contrast agent;
where the first time point, the second time point, the third time point, the fourth time point, and the fifth time point form a chronological sequence.

21. The method of claim 1, wherein the examination region is the human liver or is part of the human liver.

22. The method of claim 1, wherein each representation ^{T}R_{p+q}* generated by the machine-learning model is returned to the machine-learning model in order to generate a representation ^{T}R_{p+q+1}*.

23. The method of claim 1, wherein the representations ^{T}R₁ to ^{T}Rₙ are CT images, MRI images or ultrasound images.
